(19) United States
(12) Patent Application Publication
(10) Pub. No.: US 2008/0021547 A1
(43) Pub. Date: Jan. 24, 2008

(54) Tissue compatible heart valve sewing ring

(76) Inventor: Jim A. Davidson, San Juan Capistrano, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/491,593

(22) Filed: Jul. 24, 2006

(51) Int. Cl. A61F 2/24

(52) U.S. Cl. 623/2.41

(57) ABSTRACT

A prosthetic heart valve sewing ring that is highly compatible with surrounding tissue by being made of a material that has physical properties similar to the surrounding tissue. The present invention provides an “iso-elastic” sewing ring, or one having an elasticity similar to that of soft tissue. Exemplary materials include silicone, polyurethane, polyurethane copolymers, rubber, and other hemocompatible and biocompatible thermoplastic elastomers. The material may have an elastic modulus between about 100 to 5000, up to 10,000 psi, and more preferably between 200 to 2000 psi, a tensile strength between about 5000 and 60,000 psi, and more particularly between about 5000 to 50,000 psi, and a tensile elongation between about 100 to 3000%, preferably between about 100 to 1000%. The sewing ring may be a fiber mat, monolithic, or may have a reinforcing fiber embedded therein to help prevent suture pull-out.

### FIELD OF THE INVENTION

The present invention relates to a heart valve sewing ring that is highly compatible with surrounding tissue and, in particular, to a sewing ring made of a material that has physical properties similar to the surrounding tissue.

### BACKGROUND OF THE INVENTION

Heart valve disease continues to be a significant cause of morbidity and mortality, resulting from a number of ailments including rheumatic fever and birth defects. Recent statistics show that valvular heart disease is responsible for nearly 20,000 deaths each year in the United States, and is a contributing factor in approximately 42,000 deaths. Currently, the primary treatment of aortic valve disease is valve replacement. Worldwide, there are approximately 300,000 heart valve replacement surgeries performed annually.

Prosthetic valves attach to the patient's fibrous heart valve annulus, with or without the leaflets being present. Replacement heart valves, whether mechanical or bioprosthetic, typically utilize sewing rings or cuffs for attachment of the valve within the annulus. Sewing rings are generally composed of silicone, expanded PTFE, woven polyester fabric (e.g., polyethylene terepthalate or Dacron), silk, Prolene, and other fiber or fabric materials or combinations of these materials. The most common sewing ring construct is a silicone inner member covered with woven fabric. These materials sometimes induce the surrounding annular tissue to thicken or proliferate (hyperplasia or pannus formation). Sewing rings can also trigger short-term thrombosis and occlusion as well as the longer-term occlusion resulting from tissue hyperplasia and still longer-term atherosclerosis.

In general, sewing rings for prosthetic heart valves have performed adequately, at least relative to other complications associated with prosthetic valve implantation. However, there remains a need for improved sewing rings that minimize the host response, inflammation, and hyperplasia of surrounding tissue.

### SUMMARY OF THE INVENTION

The present invention provides a sewing ring that is highly compatible with surrounding tissue and reduces the formation of pannus. The sewing ring is formed from an “iso-elastic” polymeric material having an elastic modulus between about 100 to 10,000 psi, a tensile strength between about 5000 and 60,000 psi, and a tensile elongation between about 100 to 3000%. The material desirably has an elastic modulus between about 200 to 2000, a tensile strength between about 5000 to 50,000 psi, and a tensile elongation of between about 100 to 1000%. The sewing ring may comprise the material and at least one reinforcing fiber embedded within, such as a fiber of polypropylene. Alternatively, the sewing ring comprises a plurality of fibers formed in a mat. Desirably, the material is a polyurethane.

### BRIEF DESCRIPTION OF THE DRAWINGS

FIG. 1 is a perspective view of a prosthetic heart valve having a sewing ring of the present invention; and

FIG. 2 is a radial cross-sectional view through the exemplary sewing ring seen in FIG. 1.

### DESCRIPTION OF THE PREFERRED EMBODIMENTS

The present invention provides a sewing ring or suture cuff for prosthetic heart valves that is highly compatible with surrounding tissue. It should be understood that the tissue with which the sewing rings are compatible is healthy tissue, as opposed to calcified or stenotic tissue. The elasticity of heart tissue can vary depending on the specific tissue (myocardial, aortic wall, etc.). But in all cases, it is very elastic and stretchy compared to the various materials currently used for sewing ring construction.

Often, the leaflets or annulus of a patient's heart become highly calcified leading to the need for a replacement valve. Once implanted, the healthy tissue around the annulus sometimes exhibit hyperplasia, or proliferation, resulting from the response of local blood vessels to any compatible structure. The stiffness of conventional materials for sewing rings, such as fabric and expanded PTFE, are many tens to hundreds times the stiffness of the host tissue to which they are engaged. A local stress concentration due to the mis-match of stiffness (compliance) properties has been identified as the one of the causes of such hyperplasia. Excess tissue proliferation, including excess pannus, can interfere with proper functioning of the prosthetic heart valve.

The present invention provides an “iso-elastic” sewing ring. This term refers to an elasticity similar to that of soft tissue, in contrast to the very stiff prolene, nylon, silk, Dacron, and even PTFE (Teflon). It should be understood, however, that any polymeric fiber or material that has such physical properties is encompassed by the present invention. Suitable general classes of polymers include silicone, polyurethane, polyurethane copolymers, rubber, and other hemocompatible and biocompatible thermoplastic elastomers. A more preferred list includes polyurethane polymers and co-polymers; polyetherurethane; polycarbonateurethane, with or without silicone and their copolymers; polystyrens and their copolymers; polystyrene isobutylene and polystyrene isobutylene-styrene, and their copolymers; isobutylene and copolymers; and silicone-based polymers blended or polymerized with other appropriate polymers to increase tensile strength and meet the compliance and other material properties of the present invention. It should be noted that aside from silicone, none of these general classes of materials have been used in sewing rings, and the silicone that is used falls outside the iso-elastic material parameters described herein.

The present invention is a prosthetic heart valve sewing ring formed from any of the above-listed polymeric materials having an elastic modulus between about 100 to 5000, up to 10,000 psi, and more preferably between 200 to 2000 psi. The material also has a tensile strength between about 5000 and 60,000 psi, and more particularly between about 5000 to 50,000 psi. Finally, the tensile elongation of the material is desirably between about 100 to 3000%, preferably between about 100 to 1000%.

The following table compares the properties of elastic modulus (stiffness), tensile strength, and tensile elongation, for arterial tissue, the preferred “iso-elastic” material of the present invention, and several conventional sewing ring materials. It should be noted that there are, of course, various formulations of some of the broad classes of materials listed (e.g., “silicone”), and that the physical properties cited are exemplary only.

TABLE I
SEWING RING MATERIAL PROPERTY COMPARISON

| | Property | | |
|---|---|---|---|
| | | ULTIMATE | |
| | | TENSILE | TENSILE |
| | ELASTIC | STRENGTH | ELONGATION |
| Material | MODULUS (psi) | (psi) | (%) |
| Arterial Tissue | 200–2000 | n/a | n/a |
| (estimated) | (1.4–14 MPa) | | |
| Iso-Elastic | 100–10,000 | 5000–60,000 | 100–3000 |
| | (0.7–69.0 MPa) | (34.5–414 | |
| | | MPa) | |
| Silicone | 15–300 | 580–1813 | 600–1300 |
| | (0.1–2.1 MPa) | (4.0–12.5 MPa) | |
| Dacron | 406,000–2 · 10⁶ | 8,000–100,000 | 50–150 |
| | (2,800–13,800 MPa) | (55–690 MPa) | |
| Silk | 1.2 · 10⁶ | 85,000 | 8 |
| | (8274 MPa) | (586 MPa) | |
| Polypropylene | 322,000 (2200 MPa) | 81,000 | 26 |
| | | (558 MPa) | |

The above table illustrates the ranges of possible values for relevant physical properties of the iso-elastic materials of the present invention versus conventional sewing ring materials. It is important to understand that any polymer having the qualities described herein may be a candidate for an iso-elastic sewing ring. For example, silicone covered with woven fabric is typically used in conventional sewing rings, albeit in a form that is not iso-elastic vis-à-vis the surrounding tissue. Therefore, the table describes property ranges for the class of silicone currently used in sewing rings. Similarly, property ranges are provided for the type of Dacron that is present used for sewing rings. However, these materials might conceivably be modified to fall within the exemplary ranges for an iso-elastic material. Therefore, the comparison in the table should not be construed to exclude any of these materials per se, only these materials having properties typical in current sewing rings.

It is important to note from the table that most of the currently materials used for heart valve sewing rings are hundreds of times stiffer than arterial tissue. Only the softer conventional sewing ring materials like silicone come close to the compliance of arterial tissue. And none of these materials have the combined physical properties of the iso-elastic materials proposed herein.

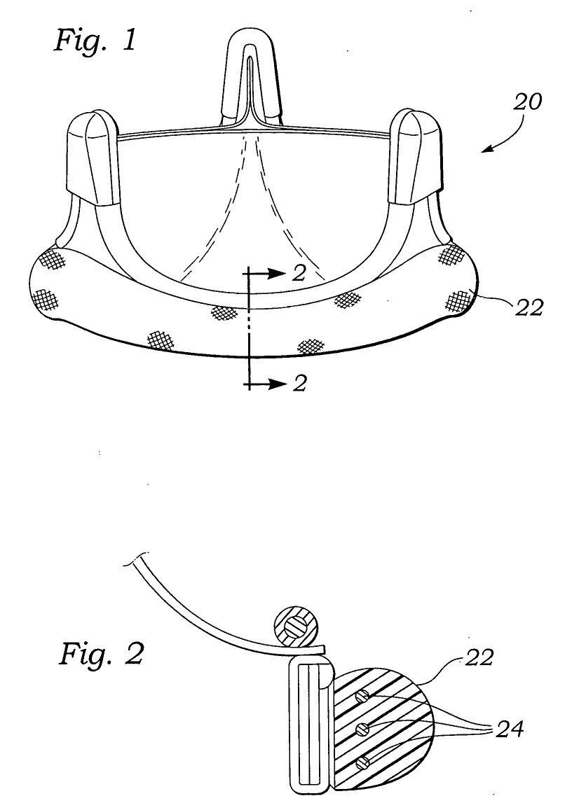

FIG. 1 illustrates an exemplary heart valve 20 in perspective view having a sewing ring 22 of the present invention. As mentioned, the heart valve 20 may have flexible leaflets, such as bioprosthetic tissue, or may be a mechanical valve with rigid leaflets. The sewing ring 22 can be a planar, annular construct, or have an undulating peripheral shape as shown.

The material of the sewing ring 22 is seen in cross-section in FIG. 2. The sewing ring 22 can have a solid cross-section section as shown, or can be provided with open cells to encourage tissue ingrowth. One embodiment is a fibrous mat formed from fibers having the material properties described herein. Some tissue ingrowth is desirable to more rapidly anchor the prosthetic heart valve to the annulus. However, the material of the sewing ring 22 is iso-elastic vis-à-vis the surrounding tissue, and therefore adverse reactions with surrounding tissue and thus excess tissue overgrowth is minimized. The sewing ring 22 attaches to the valve body in a similar manner as current sewing rings, as known to those skilled in the art. For example, sutures may attach the sewing ring 22 to the valve body, or a fabric-covered portion thereof.

It should be noted that the sewing ring 22 has no outer fabric covering, which is the case with current sewing rings, and is desirably monolithic. To help anchor the valve to surrounding tissue, fabric or fibers 24 such as polypropylene sutures may be embedded within the sewing ring 22. These fibers do not contact the surrounding tissue, and therefore do not reduce the tissue compatibility of the sewing ring 22. The fibers 24 do, however, provide reinforcing strength to the sewing ring 22 to help prevent suture pull-out. Due to the greater elasticity of the sewing ring 22 material, the density of the fabric/fiber 24 can be relatively great.

To further help reduce tissue/sewing ring mis-match, iso-elastic sutures such as those disclosed in U.S. Pat. No. 6,197,043 to Davidson are utilized. The disclosure of U.S. Pat. No. 6,197,043 is incorporated herein to the extent it discloses alternative “iso-elastic” materials.

The inventive sewing ring 22 can also be treated with anti-thrombotic agents such as heparin, hyaluronan, phosphorylcholine, platelet factors, peptides, and other proteins. Such agents can be impregnated or processed into the polymer sewing ring material as well as being coated on or added to the exterior surface to improve hemocompatibility.

While the invention has been described in its preferred embodiments, it is to be understood that the words which have been used are words of description and not of limitation. Therefore, changes may be made within the appended claims without departing from the true scope of the invention.

What is claimed is:

1. A prosthetic heart valve sewing ring formed from a polymeric material having an elastic modulus between about 100 to 10,000 psi, a tensile strength between about 5000 and 60,000 psi, and a tensile elongation between about 100 to 3000%.

2. The sewing ring of claim 1, wherein the material has an elastic modulus between about 200 to 2000.

3. The sewing ring of claim 2, wherein the material has a tensile strength between about 5000 to 50,000 psi.

4. The sewing ring of claim 3, wherein the material has a tensile elongation of between about 100 to 1000%.

5. The sewing ring of claim 1, wherein the material has a tensile strength between about 5000 to 50,000 psi.

6. The sewing ring of claim 5, wherein the material has a tensile elongation of between about 100 to 1000%.

7. The sewing ring of claim 1, wherein the material has a tensile elongation of between about 100 to 1000%.

8. The sewing ring of claim 1, wherein the sewing ring comprises the material and at least one reinforcing fiber embedded within.

9. The sewing ring of claim 8, wherein the reinforcing fiber is polypropylene.

10. The sewing ring of claim 1, wherein the sewing ring comprises a plurality of fibers formed in a mat.

11. The sewing ring of claim 1, wherein the material is a polyurethane.
